(19) United States
(12) Patent Application Publication
(10) Pub. No.: US 2008/0299643 A1
(43) Pub. Date: Dec. 4, 2008

(54) Systems and Methods for Large-Scale Production and Harvesting of Oil-Rich Algae

(76) Inventors: Everett E. Howard, Fellsmore, FL (US); Gary A. Alianell, Villa Park, CA (US); Thomas J. Riding, West Melbourne, FL (US); Peter J. Barile, Palm Bay, FL (US); Tyler R. Foster, Melbourne Beach, FL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/959,417

(22) Filed: Dec. 18, 2007

Related U.S. Application Data
(63) Continuation of application No. 11/728,297, filed on Mar. 15, 2007.
(60) Provisional application No. 60/782,564, filed on Mar. 15, 2006.
(60) Provisional application No. 60/825,464, filed on Sep. 13, 2006.
(60) Provisional application No. 60/825,592, filed on Sep. 14, 2006.

(51) Int. Cl. C12N 1/20; C12M 1/00

(52) U.S. Cl. 435/252.1; 435/289.1

(57) ABSTRACT

Systems and methods for the growing of microorganisms such as algae, yeast, and bacteria are described. Seed fermentation units are associated with final fermentation ponds in various arrangements. Continuous, semicontinuous, fed batch, and batch modes of operation of the seed and final fermentations are included. Harvest methods for the cellular material and related products are described.

### RELATED APPLICATIONS

This application is a continuation of U.S. application Ser. No. 11/728,297, filed Mar. 15, 2007, entitled SYSTEMS AND METHODS FOR LARGE-SCALE PRODUCTION AND HARVESTING OF OIL-RICH ALGAE, which is hereby expressly incorporated by reference in its entirety. This application claims benefit of priority under 35 U.S.C. §119(e) of provisional applications 60/782,564 filed Mar. 15, 2006, 60/825,592, filed Sep. 14, 2006, and 60/825,464, filed Sep. 13, 2006, which are hereby incorporated by reference in their entireties.

### FIELD OF THE INVENTION

The present invention generally relates to microorganism growth, and in particular to improved growth and harvesting for a commercially desirable level of product production.

### BACKGROUND OF INVENTION

Microorganisms, depending upon the species, increase in numbers by binary fission, budding or by filamentous growth. Binary fission is the separation of an initial cell, a mother cell, into two or more daughter cells of approximately equal size. This is a very common method of multiplication.

Budding division involves the asymmetric creation of a growing bud, on the mother cell. The bud increases in size and eventually is severed from the mother cell. After division is complete, the mother cell reinitiates the process by growing another bud. Yeast and some bacteria (e.g., Caulobacter) use this form of division. Filamentous growth is characterized by the formation of long, branching, non-divided filaments, containing multiple chromosomes. As growth proceeds, the filaments increase in length and number. Streptomyces species and many molds grow in this manner.

A desirable type of growth is binary fission. When grown in liquid medium, bacterial cultures progress through several distinguishable phases, which can be characterized by plotting the logarithm of the cell number versus time. A typical growth curve has four phases of growth, including lag phase, exponential growth phase (also termed balanced growth), stationary phase and death phase; an exemplary growth curve is illustrated in FIG. 1.

Typically, when an organism is inoculated into fresh medium, it needs to adapt to the new nutrients available, synthesize RNA and protein, and finally replicate its DNA before starting division. These processes take time, during which there is generally no net increase in cell numbers, which is characteristic of lag phase (1).

With continued reference to FIG. 1, once the appropriate enzymes for growth in a particular medium have been expressed, the cells begin to multiply. This period of maximal division can last for several hours or days, depending upon the organism, and is called the log or exponential growth phase (2).

Eventually the increase in cell number ceases, either because cells stop dividing or the rate of division equals the rate of cell death, resulting in a stationary phase (3). This is usually caused by limitation of a nutrient or an accumulation of a toxic waste product. Depending on the bacterium, a stationary phase can last for several hours to many days.

A typical growth curve can also include a death phase (4). An exponential decrease in the number of organisms due to cell death occurs during this phase. Some microorganisms never experience a death phase or it is greatly delayed due to their ability to survive for long periods without nutrients.

Factors that affect growth include, for example: temperature, pH, oxygen concentration, nutrient concentration, salt concentration, culture density, energy input (e.g., sunlight), carbon dioxide concentration, pressure, liquid depth, and degree of shear.

Current algal growth methods include photo-bio-reactors which approach laboratory conditions with high yield but typically have high capital cost. Other growth methods can include ponds that represent a partially controlled natural environment with the advantage of low capital cost, but typically carry the disadvantage of low yield.

Embodiments of the present invention also relate to methods for continuous harvest of microorganisms on a large scale. Because there can be numerous pools, each capable of being seeded from a sterile or nonsterile seed fermentation system, the growth cycle can be offset between each pool such that there can always be at least one pool ready for harvest each day.

One example of a commercially desirable product, is demonstrated by the increasing interest in bio-diesel as an alternative to petro-diesel. Such interest has led many of those skilled in the art to investigate the possibility of growing more oilseed crops as a solution to the problem of reduced future petroleum production. There are two problems with this approach: first, this would displace the food crops grown to feed mankind and second, traditional oilseed crops are not the most productive or efficient source of vegetable oil.

Micro-algae are being considered as an alternative. Such algae are, by a factor of 8 to 25 for palm oil and a factor of 40 to 120 for rapeseed, the highest potential energy-yield temperate vegetable oil crop. Micro-algae are the fastest growing photosynthesizing organisms. They can complete an entire growing cycle every few days.

The production of algae to harvest oil for biodiesel has not been undertaken on a commercial scale, but efforts to investigate feasibility are underway. In addition to the benefits of high yield, utilizing algae does not compete with agriculture for food, requiring neither farmland nor fresh water.

### SUMMARY OF THE INVENTION

Embodiments of the present invention are directed to methods of growing microorganisms such as algae, yeast, and bacteria in a pool or open tank. Embodiments provide relatively low cost and low engineering requirements. Embodiments further provide manufacturing methods for large-scale microbial growth for production of a commercially desirable product or components of a commercial product.

Yet further, embodiments of the present invention are directed to controlled continuous cultivation processes for the growth of large volumes of microorganisms. Large volumes of microorganisms can be beneficial when useful byproducts or the cell bodies are being collected for commercial purposes. Commercial products related to embodiments of the present invention include, but are not limited to, oils and fats for food, pharmaceutical, industrial and energy applications, as well as pigments and antioxidants useful in pharmaceuticals, medical imaging, food and industrial applications.

In an embodiment, a pond fermentation system is provided that comprises a central inoculum production area and two or more final fermentation ponds associated with the central inoculum production area, wherein the final fermentation ponds radiate outward from the central inoculum production area.

In a further aspect, the final fermentation ponds have a wedge shape.

In a further aspect, each final fermentation pond further comprises: a media addition region proximate to the central inoculum production area; and a biomass harvest region proximate to a distal end of the pond.

In a further embodiment, a fermentation system is provided that comprises: a water-impermeable container with fixed side walls and bottom, the pond further comprising a light transmitting top, a medium suitable for growth of photosynthetic microbes within said container, the medium in a volume within said container defining a culture depth, and a gas distributor for introducing gas below a surface of the medium, wherein the gas distributor is configured to permit log-phase growth within the container at a culture depth at least 5 times greater than a culture depth permitting log phase microbial growth without introduced gas.

In a further embodiment, a fermentation pond system is provided that comprises: at least one fermentation pond; a removable plastic liner; and a substantially homogenous monoculture of microorganisms.

In a further aspect, the substantially homogenous culture of microorganisms contains less than about 10% microorganisms other than those of the monoculture species.

In a further aspect, the removable plastic liner comprises polyethylene.

In a further aspect, the removable plastic liner is less than 200 mil thickness.

In a further embodiment, a fermentation pond system is provided that comprises: an elongate inoculum production area and at least two final fermentation ponds associated with said inoculum production area, wherein the at least two final fermentation ponds are located all to one side of said inoculum production area.

In a further embodiment, a fermentation pond system is provided that comprises: an elongate inoculum production area and at least two final fermentation ponds associated with the inoculum production area, wherein the at least two final fermentation ponds are located transverse to and on opposite sides of the inoculum production area.

In a further aspect, the inoculum production area further comprises a photobioreactor.

In a further embodiment, a method of operating a pond fermentation system is provided that comprises: growing an algal, microbial, or yeast culture in a first fermentation vessel; transferring 10-90% of the contents of the first fermentation vessel to a pond fermenter; refilling said first fermenter vessel with culture medium; and using the residual contents of said first fermenter vessel to inoculate the first fermenter culture.

In a further embodiment, a fermentation pond system is provided that comprises: a temperature control component, the component comprising: a temperature measurement component configured to measure a temperature within the system; and a control component for controlling the temperature in response to the measurement.

In a further aspect, the control component comprises a submerged coil.

In a further aspect, the control component comprises a jacket on at least one side wall or bottom wall of a culture container.

In a further embodiment, a method of growing a culture of a microorganism is provided that comprises: providing a pond fermentation system comprising at least one wedge-shaped fermentation pond; adding media approximately continuously to the pond in a vicinity of the most acute angle of the wedge-shaped pond; and harvesting the microorganism approximately continuously in the vicinity of an end of the pond opposite the angle.

### BRIEF DESCRIPTION OF THE DRAWINGS

Further aspects and advantages of embodiments of the present invention will become apparent from the following description which is given by way of example only and with reference to the accompanying drawings, wherein:

FIG. 1 depicts typical growth phases of a microorganism showing an initial lag phase, an exponential phase, a stationary phase, and a death phase.

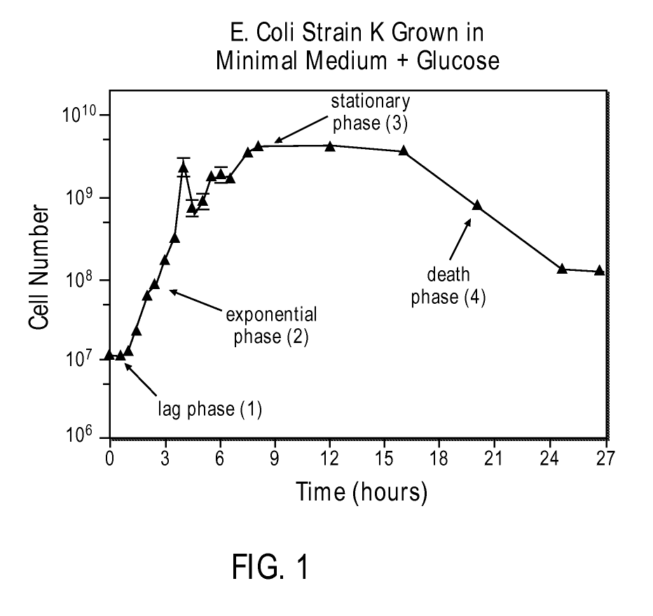

FIG. 2 is a partial diagrammatical illustration of an algae growth hybrid system.

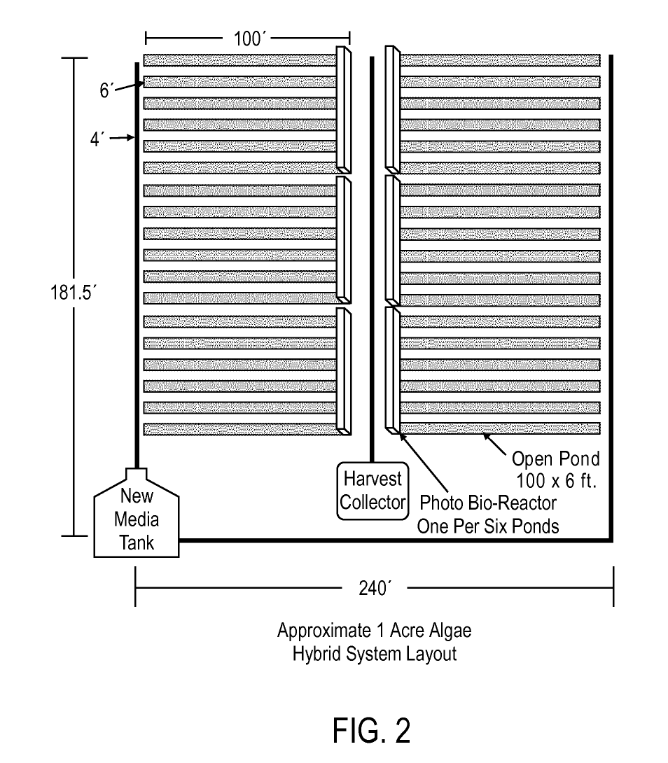

FIGS. 3-5 depict a trough-style pond fermenter with cover.

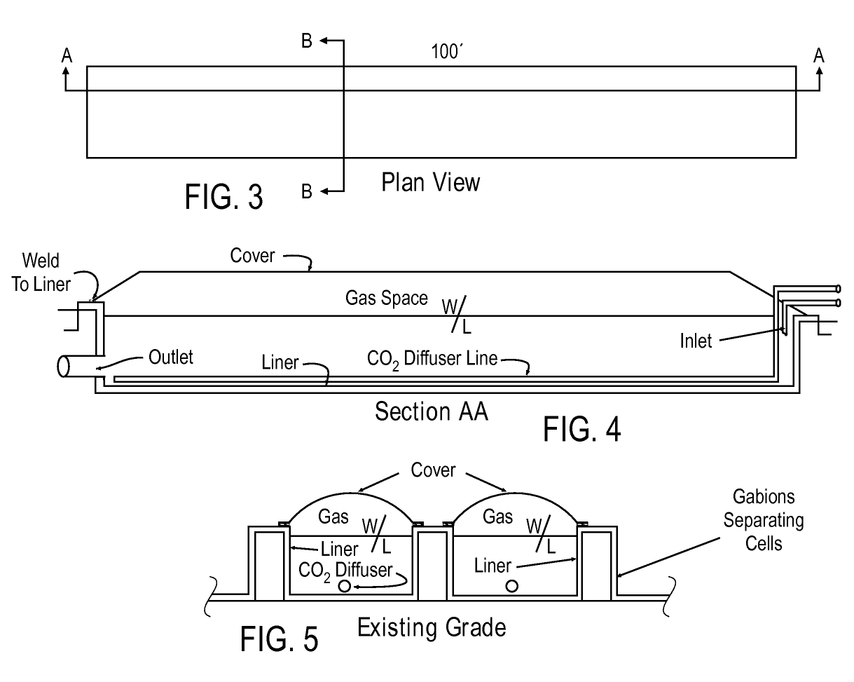

### DETAILED DESCRIPTION OF THE PREFERRED EMBODIMENT

Embodiments of the present invention will now be described more fully with reference to various alternate embodiments of the invention. It is to be understood that the invention can be embodied in many different forms and should not be construed as limited to the embodiments set forth herein. Rather, these exemplary embodiments are provided so that this disclosure can be understood by those skilled in the art.

Some embodiments of the present invention include a system for growing the microorganisms. The system can be operated in a batchwise fashion, or as a continuous or semi-continuous fermentation.

In some embodiments, a seed-stage area is located conveniently to supply a number of pond type final fermentation structures. For the purposes of this description, a fermentation pond comprises a structure built to contain a liquid where at least one horizontal dimension is more than four times the depth of liquid, the volume of liquid contained is more than 1000 L, and contains a substantially homogeneous monoculture of microorganisms. Generally, these ponds contain no more than about 10% of microorganisms that are of a different species from the monoculture species, and there is no intentional introduction of macroorganisms into the structure. The seed stage fermentation area and the final ponds can be connected via fixed piping, open trenches, closed trenches, removable piping, conduits, or other suitable means, or they can be separate, with seeding being done manually or automatically. One example of such a seed-pond arrangement comprises a central seed fermentation area and final ponds arranged as pie shaped areas emanating from this central seed fermentation area. Each quadrant or slice can be fully equipped for individual fermentation operation. A single such area can be operated alone or at the same time as other such areas. When multiple areas are operated, all can be inoculated and run at approximately the same time or the different areas can be staged to fill, be inoculated, or final at different times. In some embodiments, a facility with multiple ponds can be operated so as to have the pond fermentations ready for harvest at different times so as to achieve a steady supply of cellular material for harvest. Once the fermentation in an area is complete, or “finals,” the product can be harvested by equipment dedicated to each individual area, or with equipment that is moved from one area to another, or it can be transferred to a centralized harvesting area where harvest of the microbial cells occurs.

The final fermentation area, or “quadrant” or “slice,” can be a single, or plurality of shallow pools or open tanks. It can have a wedge or pie shape, or a different shape such as square, rectangular, elliptical, straight, curving, or other shape oriented in a radiating fashion from the central seed fermentation area. These pools can be of variable length, depth, and width within a specific pool, and one pool can vary from another. The specific dimensions can be adjusted to accommodate different ratios of inoculum to final fermentation, different growth rates of organisms, different feed strategies for different products in different organisms, different cell densities, mixing requirements, or other fermentation conditions and different product volumes. In one embodiment, the final fermentation area can be a pool with dimensions approximately 12′×50′ by 0.5 feet deep which creates a volume of about 5000 L. These dimensions can be varied as necessary to ensure sufficient sunlight penetration, adequate aeration, equipment space and circulation of nutrients for proper growth of the cells to produce the specific product desired.

In certain embodiments, a wedge-shaped fermentation pond is operated in a continuous fermentation mode. The wedge shape has particular applicability to growing photosynthetic organisms in a continuous culture. In this approach, the media, and optionally the inoculum, is added in the vicinity of the point of the wedge. As the cells grow and multiply, they move away from the point and toward the opposite wall where they are harvested. As they move in this direction, the walls of the pond diverge, providing greater surface area for the multiplying cells. This increased area provides more sunlight to the growing organisms at the same time that there are more organisms in need of sunlight. The size of the included angle of the wedge-shape determines how much the area increases as the cells move away from the inlet. This angle can be varied according to the growth of a particular organism in a particular medium under particular conditions. In such embodiments, media may be added to the pond in a media addition region. In certain embodiments, this media addition region may be proximate to or in the vicinity of a central inoculum production area. In other embodiments, this media addition region may be in the vicinity of a point or most acute angle of the wedge-shaped pond. The microorganismal biomass may be harvested in a biomass harvest region at a distal or opposite end of the pond from the point or most acute angle thereof.

Another embodiment comprises a seed fermentation area connected to final fermentation ponds arranged parallel or approximately parallel to one another, and an interconnecting distribution network between the seed fermentation and the final fermentation. A single seed fermentation area can supply all of the final ponds, or just a portion thereof, or there can be a one to one dedicated seed fermenter area to final pond association.

The seed fermentation area can be a single seed fermentation unit which supplies all of the final fermentation ponds that it is associated with. Alternatively, there can be multiple seed fermentation units within the central seed fermentation area such that individual seed fermentation units are associated with specific final fermentation ponds or a plurality of seed fermentation units are associated with each final fermentation pond. The seed fermentation unit can be a photobioreactor. A photobioreactor can be operated under sterile control. Alternatively, the seed fermentation unit can be a bioreactor without light capability or it can be a fermentation pond.

In other embodiments the seed fermentation area can be positioned next to the final fermenter ponds that it is associated with. These final fermentation ponds would extend out to one side of the seed fermentation area.

In other embodiments, the seed fermentation units can be operated in a semicontinuous mode. Less than the entire contents of a seed fermentation unit would be transferred to a final fermentation pond as inoculum, and then media would be added to the seed fermentation unit without cleaning or sterilizing the seed fermentation unit. Seed inoculum for the seed fermentation unit would be provided substantially entirely from the residue left in the seed fermentation unit from its previous cycle. This mode of operation allows for faster and more frequent filling of fermentation ponds from the seed fermentation unit as well as lower cost operation.

In various embodiments, the final fermentation ponds can be set on the ground, or elevated such as with legs, a framework, or other suitable means. The bottom of the pond can be sloped, such as to allow the pond to drain, or to aid in movement of the culture or media along the length of the pond. Alternatively, the pond can be set into the ground or have supporting walls or gabions along the sides or be made with a half-pipe construction.

In some embodiments, the walls of the pond can be insulated, jacketed, heat traced, or be bare. Alternatively, heating or cooling means, can be provided inside the fermentation pond such as with heating or cooling coils.

In some embodiments, the walls of the pond can allow transmission of light of various or specific wavelengths, or they can be opaque. The walls of the pond can allow transmission of sunlight to the fermentation culture.

The fermentation pond can include a cover. The cover can be removable or it can be permanently attached or it can be hinged. The cover can allow transmission of light, such as from sunlight or other light sources, or it can be opaque.

In another embodiment, the pond includes a replaceable liner. In some embodiments, the liner can have aeration holes; in other embodiments, the liner has no holes.

The fermentation pond can be constructed with any suitable material such as, but not limited to, stainless steel, corrosion resistant metals, plastics, ceramics, glass and elastomers. Suitable plastics and elastomers include, but are not limited to, polyethylene, polypropylene, PVC, Teflon, Tefzel, polycarbonate, acrylics, styrene, vinyl, polyurethane, rubber, buna N, nitrile, nylon, polyamide, neoprene, and combinations thereof. In one embodiment, the pond would be lined with a polyethylene material. In other embodiments, the pond would be lined with polypropylene or PVC. In another embodiment, a carbon steel trough can be lined with plastic, PVC, polyethylene, or polypropylene. In other embodiments the pond or the trough can be coated with polyethylene or other non-water-permeable coating.

Contamination of the pond with exogenous microorganisms can be controlled through media and fermentation conditions as well as with covers installed over the pond. Such covers can also prevent contamination with leaves, tweaks, sand, and other debris. Such covers can be removable or permanently affixed or hinged.

In another embodiment, the operation of the final fermentation ponds includes only surface “aeration.” The use of the term “aeration” within this description is meant to encompass all forms of delivery of a gas to the cells of the culture in the fermenter. The gas being delivered can include air, oxygen, carbon dioxide, carbon monoxide, oxides of nitrogen, nitrogen, hydrogen, inert gases, exhaust gases such as from power plants, and mixtures thereof. The gas can be pressurized or not, and can be bubbled or sparged, introduced to the surface of the fermentation culture, created in situ, or diffused through a porous or semi-permeable membrane or barrier. In other embodiments, the final fermentation ponds are aerated by bubbling or sparging gas below the surface of the liquid. In other embodiments, the final fermentation ponds are aerated by introducing the gas on one side of a porous or semi-permeable barrier with the fermentation culture on the opposite side of the barrier. In other embodiments, a combination of these methods of aeration is used.

In other embodiments, the final fermentation pond includes a mechanism for mixing the fermentation culture or media. The mechanism can be, but is not limited to, paddlewheel, propeller, turbine, paddle, or airlift. One mixing device of a single design can be used, or multiple units of a single design can be used, or multiple units of different designs can be used. The mixing unit can be used to impart directional motion to the fermentation culture, such as to move the culture further along the linear or side to side dimension of the pond, or it can be used to impart vertical movement to the culture, such as to move cells to or away from the surface, or it can be used to mix the culture in place, create shear, break up bubbles, break up aggregated masses of cells, to mix in nutrients, to bring the cells into contact with nutrients, or it can be used to do a combination of these things. Airlift can be achieved by injecting gas under high or low pressure into the pond, or by more gentle means such as by introducing gas below the surface of the pond and allowing bubbles to rise to the surface. One embodiment of an airlift system can include a pipe with one or a plurality of holes facing up, down, to the sides, or a combination of these, positioned below the surface of the pond, introducing a gas to the interior of the pipe, and allowing or forcing the air to move out through the holes. Another embodiment utilizes a chamber instead of a pipe. In different embodiments, the pipe or chamber can be affixed in one position in the fermenter, or it can be portable and be moved either between fermentations or during a fermentation. Such movement can be done manually, or automatically. Other embodiments can attach the pipe or chamber to the bottom of the pond, the side of the pond, the top of the pond, or the ground near the pond, either directly or with a support structure. In another embodiment, the fermentation pond comprises a replaceable liner where the liner includes aeration holes and gas is introduced below the liner and allowed to bubble through the culture on the other side of the liner wall. The shape of the holes used for aeration can be round or square or any other suitable shape. They can be converging or diverging, have sharp edges, have rounded edges, be of uniform size, be of differing sizes, be perpendicular to the wall of the pipe or chamber or liner, or be set at an angle to a line drawn perpendicular to the pipe, chamber, or liner.

In operation, different organisms can be grown in a variety of different media in the subject bioreactors. Examples of suitable media include, but are not limited to, Luria Broth, brackish water, water having nutrients added, dairy runoff, media with salinity of less than or equal to 1%, media with salinity of greater than 1%, media with salinity of greater than 2%, media with salinity of greater than 3%, media with salinity of greater than 4%, and combinations thereof. Nitrogen sources can include nitrates, ammonia, urea, nitrites, ammonium salts, ammonium hydroxide, ammonium nitrate, monosodium glutamate, soluble proteins, insoluble proteins, hydrolyzed proteins, animal byproducts, dairy waste, casein, whey, hydrolyzed casein, hydrolyzed whey, soybean products, hydrolyzed soybean products, yeast, hydrolyzed yeast, corn steep liquor, corn steep water, corn steep solids, distillers grains, yeast extract, oxides of nitrogen, N2O, or other suitable sources. Carbon sources can include sugars, monosaccharides, disaccharides, sugar alcohols, fats, fatty acids, phospholipids, fatty alcohols, esters, oligosaccharides, polysaccharides, mixed saccharides, glycerol, carbon dioxide, carbon monoxide, starch, hydrolyzed starch, or other suitable sources.

Additional media ingredients can include buffers, minerals, growth factors, anti-foam, acids, bases, antibiotics, surfactants, or materials to inhibit growth of undesirable cells.

The nutrients can be added all at the beginning, or some at the beginning and some during the course of the fermentation as a single subsequent addition, as a continuous feed during the fermentation, as multiple dosing of the same or different nutrients during the course of the fermentation, or as a combination of these methods.

The pH of the culture can be controlled through the use of a buffer or by addition of an acid or base at the beginning or during the course of the fermentation. In some cases, both an acid and a base can be used in different zones of the pond or in the same zone at the same or different times in order to achieve a desirable degree of control over the pH. Non-limiting examples of buffer systems include phosphate, TRIS, TAPS, bicine, tricine, HEPES, TES, MOPS, PIPES, cacodylate, MES, and acetate. Nonlimiting examples of acids include sulfuric acid, HCl, lactic acid, and acetic acid. Nonlimiting examples of bases include potassium hydroxide, sodium hydroxide, ammonium hydroxide, ammonia, sodium bicarbonate, calcium hydroxide, and sodium carbonate. Some of these acids and bases in addition to modifying the pH can also serve as a nutrient for the cells. The pH of the culture can be controlled to approximate a constant value throughout the entire course of the fermentation, or it can be changed during the fermentation. Such changes can be used to initiate or end different molecular pathways, to force production of one particular product, to force accumulation of a product such as fats, dyes, or bioactive compounds, to suppress growth of other microorganisms, to suppress or encourage foam production, to force the cells into dormancy, to revive them from dormancy, or for some other purpose.

Likewise, the temperature of the culture can in some embodiments be controlled to approximate a particular value or it can be changed during the course of the fermentation for the same or different purposes as listed for pH changes. In certain of such embodiments, a temperature control component is provided that comprises a temperature measurement component that measures a temperature within the system, such as a temperature of the medium, and a control component that can control the temperature in response to the measurement. The control component may comprise a submerged coil or a jacket on the side or bottom wall of the culture container.

Once the culture has achieved a sufficient degree of growth, the cells can be harvested. Harvest can occur directly from the pond or after transfer of the culture to a storage tank. The harvesting steps can include the steps of killing the cells or forcing them into dormancy, separating the cells from the bulk of the media, drying the cells, lysing the cells, separating the desirable components, and isolating the desired product. In some embodiments, not all of these steps are practiced together; various embodiments can combine various different steps and can also include additional steps and/or combinations of various functions into one or several steps, such that some of the steps can be combined. Additionally the steps actually practiced can be practiced in a different order than presented in this list.

Killing or forced dormancy of the cells can be accomplished by a number of means depending on the cells and the product desired. Suitable means include, but is not limited to, heating, cooling, addition of chemical agents such as acid, base, sodium hypochlorite, enzymes, sodium azide, or antibiotics.

Separation of the cell mass from the bulk of the water can be accomplished in a number of ways. Non-limiting examples include screening, centrifugation, rotary vacuum filtration, pressure filtration, hydrocycloning, flotation, skimming, sieving and gravity settling. Other techniques, such as addition of precipitating agents, flocculating agents, or coagulating agents, can also be used in conjunction with these techniques. In some cases, the desired product will be in one of the streams from a separating device and in other cases it will be in the other stream. Two or more stages of separation can be used. When multiple stages are used, they can be based on the same or a different technique. Non-limiting examples include screening of the bulk of the fermenter contents, followed by filtration or centrifugation of the effluent from the first stage.

In some cases, it will be desirable to dry the cellular material prior to further processing. For example, drying can be desired when the subsequent processing occurs in a remote location or requires larger volumes of material than are provided by a single fermentation batch, or if the material must be campaigned through to achieve more cost-effective processing, or if the presence of water will cause processing difficulties such as emulsion formation, or for other reasons not listed here. Suitable drying systems include, but are not limited to, air drying, solar drying, drum drying, spray drying, fluidized bed drying, tray drying, rotary drying, indirect drying, or direct drying.

Cell lysis can be achieved mechanically or chemically. Non-limiting examples of mechanical methods of lysis include pressure drop devices such as use of a French press or a pressure drop homogenizer, colloid mills, bead or ball mills, high shear mixers, thermal shock, heat treatment, osmotic shock, sonication, expression, pressing, grinding, expeller pressing and steam explosion. Non-limiting examples of chemical means include the use of enzymes, oxidizing agents, solvents, surfactants, and chelating agents. Depending on the exact nature of the technique being used, the lysis can be done dry, or a solvent, water, or steam can be present. Solvents that can be used for the lysis or to assist in the lysis include, but are not limited to hexane, heptane, supercritical fluids, chlorinated solvents, alcohols, acetone, ethanol, methanol, isopropanol, aldehydes, ketones, chlorinated solvents, fluorinated-chlorinated solvents, and combinations of these. Exemplary surfactants include, but are not limited to, detergents, fatty acids, partial glycerides, phospholipids, lysophospholipids, alcohols, aldehydes, polysorbate compounds, and combinations of these. Exemplary supercritical fluids include carbon dioxide, ethane, ethylene, propane, propylene, trifluoromethane, chlorotrifluoromethane, ammonia, water, cyclohexane, n-pentane, and toluene. The supercritical fluid solvents can also be modified by the inclusion of water or some other compound to modify the solvent properties of the fluid. Suitable enzymes for chemical lysis include proteases, cellulases, lipases, phospholipases, lysozyme, polysaccharases, and combinations thereof. Suitable chelating agents include, but are not limited to EDTA, porphine, DTPA, NTA, HEDTA, PDTA, EDDHA, glucoheptonate, phosphate ions (variously protonated and nonprotonated), and combinations thereof. In some cases, combinations of chemical and mechanical methods can be used.

Separation of the broken cells from the product containing portion or phase can be accomplished by various techniques. Non-limiting examples include centrifugation, hydrocycloning, filtration, floatation, and gravity settling. In some situations, it would be desirable to include a solvent or supercritical fluid, for example, to solubilize desired product, reduce interaction between the product and the broken cells, reduce the amount of product remaining with the broken cells after separation, or to provide a washing step to further reduce losses. Suitable solvents include, but are not limited to hexane, heptane, supercritical fluids, chlorinated solvents, alcohols, acetone, ethanol, methanol, isopropanol, aldehydes, ketones, and fluorinated-chlorinated solvents. Exemplary supercritical fluids include carbon dioxide, ethane, ethylene, propane, propylene, trifluoromethane, chlorotrifluoromethane, ammonia, water, cyclohexane, n-pentane, toluene, and combinations of these. The supercritical fluid solvents can also be modified by the inclusion of water or some other compound to modify the solvent properties of the fluid.

The product so isolated can then be further processed as appropriate for its desired use such as by solvent removal, drying, filtration, centrifugation, chemical modification, transesterification, further purification, or by some combination of steps.

In the final fermenter step, the fermentation ponds, can be operated in batch mode, continuous mode, or semi-continuous mode. For example, in a batch mode the pond would be filled to appropriate level with fresh and/or recycled media and inoculum. This fermentation would then be allowed to run until the desired degree of growth has occurred. At this point, harvest of the product would occur. In one embodiment, the entire fermenter contents would be harvested, then the fermenter would be cleaned and sanitized as needed and refilled with media and inoculum. In another embodiment, only a portion of the fermenter contents would be harvested, for example approximately 50%, then media would be added to refill the pond and the fermentation would continue.

Alternatively, the final fermenter step can be operated in a continuous mode. In a continuous mode, media, fresh and/or recycled, or media, fresh and/or recycled, and fresh inoculum are continuously fed to the pond while harvest of cellular material occurs continuously. In continuous operation, there can be an initial startup phase where the harvest is delayed to allow sufficient cell concentration to build up. During this startup phase, the media feed and/or inoculum feed can be interrupted. Alternatively, media and inoculum can be added to the pond and when the pond gets to the desired liquid volume, harvest commences. Other startup techniques can be used as desired to meet operational requirements and as appropriate for the particular product organism and growth medium. Where a culture is grown in a first fermentation vessel, approximately 10-90%, or 20-80%, or 30-70% of the culture may be transferred to a final fermentation pond, with the residual contents serving a starter culture for subsequent growth in the first fermentation vessel.

A continuous pond fermenter can be operated in a “stirred mode” or a “plug flow mode” or a “combination mode.” In a stirred mode, the media and inoculum are added and mixed into the general volume of the pond. Mixing devices include, but are not limited to paddlewheel, propeller, turbine, paddle, or airlift operating in a vertical, horizontal or combined direction. In some embodiments, the mixing can be achieved or assisted by the turbulence created by adding the media or inoculum. The concentration of cells and media components does not very greatly across the horizontal area of the pond. In a plug flow mode, the media and inoculum are added at one end of the pond, and harvest occurs at the other end. In the plug flow mode, the culture moves generally from the media inlet toward the harvest point. Cell growth occurs as the culture moves from the inlet to the harvest location. Movement of the culture can be achieved through means including, but not limited to, sloping the pond, mixing devices, pumps, gas blown across the surface of the pond, and the movement associated with the addition of material at one end of the pond and removal at the other. Media components can be added at various points in the pond to provide different growing conditions for different phases of cell growth. Likewise, the temperature and pH of the culture can be varied at different points of the pond. Optionally, back mixing can be provided at various points. Act mixing can be achieved through the use of mixers, paddles, baffles or other appropriate techniques.

In a combination mode, a portion of the pond will operate in a plug flow mode, and a portion would operate in a stirred mode. For example, media can be added in a stirred zone to create a “self seeding” or “self inoculating” fermentation system. The media with growing cells would move from the stirred zone to a plug flow zone where the cells would continue their growth to the point of harvest. Stirred zones can be placed at the beginning, in the middle, or toward the end of the pond depending on the effect desired. In addition to creating a self seeding fermentation, such stirred zones can be used for purposes including, but not limited to, providing a specific residence time exposing the cells to specific conditions or concentrations of particular reagents or media components. Such stirred zones can be achieved through the use of baffles, barriers, diverters, and/or mixing devices.

A semi-continuous pond fermenter can be operated by charging the pond with an initial quantity of media and inoculum. As the fermentation runs, additional media is added either continuously, or at intervals.

Methods used to clean, sanitize, and sterilize the ponds include, but are not limited to low-pressure steam, detergents, surfactants, chlorine, bleach, ozone, UV light, peroxide, and combinations thereof. In one embodiment, the pond would be rinsed with water, washed with a detergent, rinsed with water, sprayed with a bleach solution (sodium hypochlorite), and then filled with media and inoculum. In other embodiments, the pond can be filled with bleach solution and drained, the bleach solution can be neutralized with a reducing agent such as sodium thiosulfate.

In one embodiment, the pond designs of the present invention can be used for microorganisms that float, either throughout their growth cycle or only at particular points in their growth cycle. For example, some microorganisms produce oils, which being lighter than water, will cause the cell to float when present in sufficient quantity. Other organisms can trap gases which cause the organism to float. Such microorganisms can be collected off the surface of the pond, such as by rotary vacuum filtration, skimming, or flotation. In another embodiment, a continuous fermentation pond is operated with floating cells where the cells are collected off the surface of the pond. In a further embodiment, photosynthetic floating cells are collected from the surface at a harvest point while cells continue to grow and consume carbon dioxide elsewhere in the pond.

In other embodiments, the pond designs of the present invention can be used for growth of oil-producing photosynthetic microorganisms. These microorganisms can be recovered from the ponds, and the biomass used directly as a fuel, either dried or in a wet state. In another embodiment, the oil-producing photosynthetic microorganisms can be collected from the ponds and the oil can be liberated by expression, such as with an expeller press, batch press, or filter press or the oil can be solvent extracted such as with hexane, heptane, alcohols, or other solvents or supercritical fluids as described elsewhere in this description. Such extraction can be combined with mechanical or chemical cell lysis as described elsewhere in this specification.

Many modifications and other embodiments of the invention will come to the mind of one skilled in the art having the benefit of the teachings presented in the foregoing descriptions and the associated drawings. Therefore it is to be understood that the invention is not to be limited to the specific embodiments disclosed, and that modifications and alternate embodiments are intended to be included within the scope of the claims supported by this specification.

What is claimed is:

1. A pond fermentation system comprising: a central inoculum production area and two or more final fermentation ponds associated with said central inoculum production area, wherein said final fermentation ponds radiate outward from said central inoculum production area.

2. The pond fermentation system of claim 1, wherein the final fermentation ponds have a wedge shape.

3. The pond fermentation system of claim 2, wherein each final fermentation pond further comprises:
a media addition region proximate to said central inoculum production area; and
a biomass harvest region proximate to a distal end of said pond.

4. A pond fermentation system comprising: a water impermeable container with fixed side walls and bottom, the pond further comprising a light transmitting top, a medium suitable for growth of photosynthetic microbes within said container, said medium in a volume within said container defining a culture depth, and a gas distributor for introducing gas below a surface of the medium, wherein the gas distributor is configured to permit log-phase growth within the container at a culture depth at least 5 times greater than a culture depth permitting log phase microbial growth without introduced gas.

5. A fermentation pond system comprising:
at least one fermentation pond;
a removable plastic liner; and
a substantially homogenous monoculture of microorganisms.

6. The fermentation pond system of claim 5, wherein said substantially homogenous culture of microorganisms contains less than about 10% microorganisms other than those of a monoculture species.

7. The fermentation pond of claim 5 wherein the removable plastic liner comprises polyethylene.

8. The fermentation pond of claim 6 wherein the removable plastic liner is less than 200 mil thickness.

9. A pond fermentation system comprising: an elongate inoculum production area and at least two final fermentation ponds associated with said inoculum production area, wherein said at least two final fermentation ponds are located all to one side of said inoculum production area.

10. A pond fermentation system comprising: an elongate inoculum production area and at least two final fermentation ponds associated with said inoculum production area, wherein said at least two final fermentation ponds are located transverse to and on opposite sides of said inoculum production area.

11. The fermentation system of claim 1 wherein the inoculum production area further comprises a photobioreactor.

12. A method of operating a pond fermentation system comprising: growing an algal, microbial, or yeast culture in a first fermentation vessel; transferring 10-90% of the contents of said first fermentation vessel to a pond fermenter; refilling said first fermenter vessel with culture medium; and using the residual contents of said first fermenter vessel to inoculate said first fermenter culture.

13. A fermentation system comprising a temperature control component, said component comprising:
a temperature measurement component configured to measure a temperature within said system; and
a control component for controlling said temperature in response to said measurement.

14. The fermentation system of claim 11 wherein the control component comprises a submerged coil.

15. The fermentation system of claim 11 wherein the control component comprises a jacket on at least one side wall or bottom wall of a culture container.

16. A method of growing a culture of a microorganism, comprising:
providing a pond fermentation system comprising at least one wedge-shaped fermentation pond;
adding media approximately continuously to said pond in a vicinity of the most acute angle of said wedge-shaped pond; and
harvesting said microorganism approximately continuously in a vicinity of an end of said pond opposite said angle.

17. A system for growing microorganisms, comprising:
the fermentation system of claim 1; and
at least one microorganism selected from the group consisting of Pseudochlorococcum sp., Chlorococcum sp., Chlorella sp., Scenedesmus sp., Palmellococcus sp., Cylindrospermopsis sp., and Planktothrix sp.

18. The system of claim 17, further comprising an energy source.

19. The system of claim 17, further comprising a media supply.

20. The system of claim 17 wherein the energy source comprises combustion of the biomass produced by the system.

21. The system of claim 17 wherein the media comprises waste-water.

22. The system of claim 17 wherein the microorganism comprises Chlorella sp.

23. The system of claim 17 wherein the microorganism comprises Pseudochlorococcum sp.

24. A method for growing microorganisms, comprising:
adding media to the fermentation system of claim 1;
sterilely inoculating the fermentation system with a microorganism selected from the group consisting of Pseudochlorococcum sp., Chlorococcum sp., Chlorella sp., Scenedesmus sp., Palmellococcus sp., Cylindrospermopsis sp., and Planktothrix sp.;
monitoring at least one pre-determined parameter of the culture, selected from the group consisting of: pH, temperature, O₂ concentration, CO₂ concentration, NO₃⁻/PO₄³⁻ levels, conductivity, turbidity; and
harvesting at least a part of the culture when the culture exceeds at least one pre-determined parameter selected from the group consisting of: pH, temperature, O₂ concentration, CO₂ concentration, NO₃⁻/PO₄³⁻ levels, conductivity, and turbidity.

25. The method of claim 24 wherein the media comprises waste-water.

26. The method of claim 24 wherein the microorganism comprises Chlorella sp.

27. The method of claim 24 wherein the microorganism comprises Pseudochlorococcum sp.

28. An apparatus for growing microorganisms, comprising:
a plurality of the systems of claim 17, wherein each of the plurality of systems are fluidly-connected to a single bioreactor.
